(19) United States
(12) Patent Application Publication
(10) Pub. No.: US 2001/0023351 A1
(43) Pub. Date: Sep. 20, 2001

(54) Skin abrasion system and method

(76) Inventors: George J. Eilers, Evergreen, CO (US); Steven H. Johnson, Lakewood, CO (US)

Correspondence Address: ERIC B. MEYERTONS, CONLEY, ROSE & TAYON, P.C., P. O. BOX 398, AUSTIN, TX, 78767-0398, US

(21) Appl. No.: 09/728,426

(22) Filed: Dec. 1, 2000

Related U.S. Application Data
(60) Provisional application No. 60/203,541, filed on May 10, 2000.
(60) Provisional application No. 60/203,539, filed on May 10, 2000.
(60) Provisional application No. 60/168,417, filed on Dec. 1, 1999.

(51) Int. Cl. A61B 17/50

(52) U.S. Cl. 606/131

(57) ABSTRACT

Rounded particles may be used as an abrasive during a microdermabrasion procedure. Rounded particles may be propelled against skin within a treatment area to treat the skin. The rounded particles may abrade portions of the skin within the treatment area. The rounded particles used in a microdermabrasion procedure may be mixed with other abrasives and materials. The rounded particles may be glass beads. The rounded particles may be coated with other materials such as coloring agents, vitamins, lotion, or antibacterial agents.

### PRIORITY CLAIM

This application claims priority to U.S. Provisional Patent Application 60/203,541 filed May 10, 2000, to U.S. Provisional Patent Application 60/203,539 filed May 10, 2000, and to U.S. Provisional Patent Application 60/168,417 filed Dec. 1, 1999.

### BACKGROUND OF THE INVENTION

1. Field of the Invention

The present invention generally relates to skin abrasion procedures. More particularly, an embodiment of the invention relates to the use of rounded particles during a microdermabrasion procedure.

2. Description of the Related Art

A microdermabrasion procedure may be used to treat skin. Abrasive particles may be propelled against a treatment area during a microdermabrasion procedure. The abrasive particles may abrade and remove a portion of the skin. A microdermabrasion procedure may be used in place of or in conjunction with a laser resurfacing procedure or a chemical peel.

In one type of microdermabrasion procedure, a vacuum may be used to draw abrasive particles across a treatment area. The vacuum may serve to propel the abrasive particles and to remove abraded skin and abrasive particles to a waste receptacle. Alternatively, the abrasive particles may be propelled against the treatment area by a compressed gas. A vacuum may be used to draw abraded skin and abrasive particles to a waste receptacle.

A microdermabrasion procedure may be used to remove the epidermal skin layer, or selected portions of the epidermal skin layer, such as the stratum corneum. Removal of all or selected portions of the epidermal layer from a treatment area may stimulate underlying skin tissue. Stimulation of underlying skin tissue may serve to freshen or tone the skin. A microdermabrasion procedure may also be used to remove portions of the dermal skin layer from a treatment area. Removing portions of the dermal skin layer may remove undesired skin pigmentation or blend the color of the treatment area to more closely match the skin pigmentation of adjacent skin.

A microdermabrasion procedure may be used to freshen or tone the skin, to treat wrinkles, such as aging wrinkles, to treat stretch marks, and/or to treat skin blemishes. A microdermabrasion procedure may be used to treat skin blemishes that include, but are not limited to certain forms of keratoses, acne, scar tissue, calluses, melasma, hyper-pigmentation, photo or sun damaged skin, and tattoos.

During a microdermabrasion procedure, a handpiece of a microdermabrasion machine may be guided over a treatment area. A vacuum may be used to draw abrasive particles from a supply receptacle across the treatment area. The particles may abrade and remove portions of the skin. The vacuum may draw the abrasive particles and removed skin into a waste receptacle. The vacuum may typically range from about 10 to 50 centimeters of mercury. The vacuum may stretch the skin and cause local vasodilation.

In addition to the vacuum, a compressed gas may be used to propel the abrasive particles against the treatment area to improve the abrasive effect of the particles. The use of vacuum and compressed gas to propel the abrasive particles against the skin may allow for greater abrasion of the skin than can be obtained using only vacuum to propel the abrasive particles.

A microdermabrasion machine may be used during a microdermabrasion procedure. U.S. Pat. No. 5,810,842 issued to Di Fiore et al., and U.S. Pat. No. 5,037,432 issued to Molinari, describe microdermabrasion machines. Each of these patents are incorporated by reference as if fully set forth herein.

Aluminum oxide is typically used as the abrasive during a microdermabrasion procedure. Synonyms for aluminum oxide include alumina, aluminum trioxide, and corundum powder. The aluminum oxide used in a microdermabrasion procedure may be in the form of aluminum oxide particles. The aluminum oxide particles may be irregularly shaped. The aluminum oxide particles may have sharp edges. An electrode process may be used to form aluminum oxide particles having sharp edges. Sharp edged particles may have good abrasive properties when used as an abrasive in a microdermabrasion procedure.

Aluminum oxide particles may be sieved so that the aluminum oxide particles are predominantly within a desired size range. Mesh screens may be used to isolate aluminum oxide particles with a desired effective diameter size range. The effective diameter size range for sharp edged aluminum oxide particles useful for microdermabrasion procedures may be between about 50 microns and 180 microns. Commercially available aluminum oxide particles suitable for use in a microdermabrasion procedure typically include a percentage of fines. Fines are particles that are significantly smaller than the desired size range of particles. Fines may have effective diameters less than about ten microns in size. The presence of fines in the abrasive particles used for microdermabrasion procedures may cause health problems and may cause problems with microdermabrasion equipment. Aluminum oxide particles may be processed to remove fines, but aluminum oxide particles that have minimal or no fines may be prohibitively expensive.

Fines are undesirable because fines may be dispersed in the air during transfer of abrasive from one container to another. Fines may also become airborne during a microdermabrasion procedure. The fines may be visible as a fine smoke-like dust when airborne. The generation of airborne fines may be problematic because fines have a tendency to bind with infectious materials. The fines may transport such infectious materials through the air.

Fines may also cause problems with a microdermabrasion machine. Fines may cause abrasive particles within a microdermabrasion machine to clump. Clumped abrasive particles may plug conduits within a microdermabrasion machine and stop the machine from functioning. Fines may also cause excessive wear of parts within a microdermabrasion machine. The small size of fines may allow fines to pass through filters that protect the vacuum pump of a microdermabrasion machine. To prevent excessive machine wear, frequent maintenance and replacement of parts of a microdermabrasion machine may be required.

### SUMMARY OF THE INVENTION

Rounded particles may be used as an abrasive in a microdermabrasion procedure. Rounded particles may serve as a polishing or renewing agent for the skin. Rounded particles may abrade portions of skin within a treatment area during a microdermabrasion procedure.

In certain embodiments, an abrasive used in a microdermabrasion procedure may be a mixture of rounded particles and other abrasives. In an embodiment, the abrasive is a mixture of rounded particles and sharp-edged particles. The sharp-edged particles may be, but are not limited to sand, glass, or aluminum oxide particles having sharp edges. In a mixture of rounded particles and sharp-edged particles, the rounded particles may inhibit clumping of the sharp-edged particles. The rounded particles may be hollow particles, such as, but not limited to hollow glass beads. Also, an abrasive that is a mixture of rounded particles and sharp-edged particles may be less expansive than an abrasive including only sharp-edged particles, such as sharp-edged aluminum oxide particles; yet the abrasive mixture may have substantially the same or similar abrasive characteristics as the abrasive including only the sharp-edged particles.

Rounded particles used as an abrasive in a microdermabrasion procedure may be mixed or coated with other materials. The other materials may include, but are not limited to, lotions, antibacterial agents, coloring agents, and vitamins.

In embodiments, rounded particles used during a microdermabrasion procedure may be substantially spherical in shape. In other embodiments, the rounded particles may have non-spherical geometries with rounded edges. The rounded particles may have an effective particle diameter size range that allow the particles to pass through a sieve having a particular mesh size, but not pass through a sieve having a smaller mesh size. The rounded particles may have a narrow particle diameter size distribution range. Rounded particles, such as glass beads, may be commercially obtained in several different narrow particle diameter size distribution ranges. The particle diameter size distribution range of the rounded particles may be between about 25 microns and about 325 microns, or between about 50 microns and about 250 microns, or between about 100 microns and about 200 microns. A narrow particle diameter size distribution range may be preferred over a broad particle diameter size distribution range. For example, glass beads having a particle diameter size distribution range from about 90 microns to about 150 microns may be used in a microdermabrasion procedure. A narrow particle diameter size distribution range of rounded particles may produce a more uniform abrasive effect in a treatment area than can be obtained when using a broad particle diameter size distribution range of abrasive particles.

Glass beads may be the abrasive used in a microdermabrasion procedure. Several characteristics of glass beads make glass beads well suited for use as the abrasive in a microdermabrasion procedure. Glass beads may be commercially available in distribution ranges that are narrower than the distribution ranges available for aluminum oxide particles. Glass beads may be commercially available at a lower price than aluminum oxide particles. Glass beads have approximately half the density of aluminum oxide particles. Because the density of glass beads is less than the density of aluminum oxide particles, it may cost less to ship a given volume of glass beads than it would to ship the same volume of aluminum oxide particles. Also, commercially purchased glass beads contain few particles that are small enough to become airborne during normal use and handling. Using glass beads instead of at least some of the aluminum oxide particles may substantially reduce and/or eliminate the presence of fines and small particles in the abrasive. The substantial reduction and/or elimination of fines may avoid the harmful effects of small particles on the microdermabrasion machinery, on the operators of the machinery, and on the patients undergoing microdermabrasion procedures.

The abrasive effect on the skin of rounded particles may be different than the abrasive effect on the skin of irregularly shaped aluminum oxide particles. When only a vacuum is used to draw abrasive particles across the skin, rounded particles appear to have less effect on the skin than do irregularly shaped aluminum oxide particles. Using a vacuum to draw rounded particles across a treatment area of skin may be well suited to toning and refreshing the skin. When a compressed gas is used to propel abrasive particles against the skin at a selected velocity, rounded particles appear to have less effect on the skin than do irregularly shaped aluminum oxide particles.

When a compressed gas or air is used to propel abrasive particles against the skin at a given pressure, glass beads may be propelled against the skin at a greater velocity than irregularly shaped aluminum oxide particles of the same general size because of the lighter density of the glass beads. The faster velocity of the glass beads may allow the glass beads to have a similar abrasive effect to the effect produced by irregularly shaped aluminum oxide particles propelled at the same operating pressure.

An advantage of the use of rounded particles during a microdermabrasion procedure is that rounded particles typically are not embedded in the skin during the procedure. Substantially all of the rounded particles may be removed from a treatment area by the vacuum that draws the particles from the treatment area to a waste receptacle. When sharp-edged aluminum oxide particles are used during a microdermabrasion procedure, a portion of the particles may become embedded in the skin during the procedure.

Another advantage of the use of rounded particles is that commercially available rounded particles have few or no fines. The absence of fines may eliminate problems of airborne particles during the setup and use of a microdermabrasion machine. The absence of fines may extend the life of the microdermabrasion machine and decrease the expenses associated with maintenance of the microdermabrasion machine.

Another advantage of using rounded particles during a microdermabrasion procedure may be that the rounded particles produce a more controllable and a more desirable effect on the treated skin than is obtainable with the use of irregularly shaped aluminum oxide particles. Rounded particles are typically less abrasive than are irregularly shaped aluminum oxide particles. The milder abrasive characteristics of the rounded particles may allow an operator of a microdermabrasion machine to have greater control of the abrasive effect produced by the particles in a treatment area.

Another advantage of using rounded particles may be that the rounded particles are more cost effective than irregularly shaped aluminum oxide particles. The use of rounded particles, such as glass beads, may be more cost effective than the use of aluminum oxide particles because of product cost. Also, shipping a volume of rounded particles, such as glass beads, may cost less than shipping an equal volume of irregularly shaped aluminum oxide particles. Rounded particles that have approximately the same size range distribution as the allowable size range of aluminum oxide particles may be used in existing microdermabrasion machines without the need to modify the existing microdermabrasion machines. Further advantages of using rounded particles within a microdermabrasion machine may include that the round particles are sturdy, durable, light weight, simple, efficient, safe, easily obtainable, reliable and inexpensive; yet the rounded particles may also be easy to handle, install and use with a microdermabrasion machine.

### BRIEF DESCRIPTION OF THE DRAWINGS

Further advantages of the present invention will become apparent to those skilled in the art with the benefit of the following detailed description of embodiments and upon reference to the accompanying drawings in which:

FIG. 1 shows a cut-away view of a portion of a handpiece of a microdermabrasion machine during a microdermabrasion procedure;

FIG. 2 is a diagrammatic view of a microdermabrasion machine;

FIG. 3 is a perspective view of a microdermabrasion machine;

FIG. 4 shows a perspective view of an embodiment of a microdermabrasion machine;

FIG. 5 is a schematic representation of a microdermabrasion machine that includes a compressor that is activated by an electrically operated activation mechanism located on a handpiece;

FIG. 6 is a schematic representation of a microdermabrasion machine that includes a compressor that is activated by a vacuum operated pneumatic activation mechanism located on a handpiece;

FIG. 7 is a schematic representation of a microdermabrasion machine that includes a compressor that is activated by a positive pressure operated pneumatic activation mechanism located on a handpiece;

FIG. 8 is a perspective view of a handpiece that includes an electrically operated compressor control mechanism;

FIG. 9 is a perspective view of a handpiece that includes a pneumatically operated compressor control mechanism;

FIG. 10 shows an exploded view of a body of a pneumatically controlled handpiece;

FIG. 11 shows a top view of an embodiment of a tip for a handpiece with pneumatic control;

FIG. 12 shows a perspective view of a tip body for an embodiment of an electrically controlled handpiece;

FIG. 13 shows a perspective view of a tip body for an embodiment of a pneumatically controlled handpiece; and

FIG. 14 is a plan view of a metallic insert tube for a handpiece.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF THE PREFERRED EMBODIMENTS

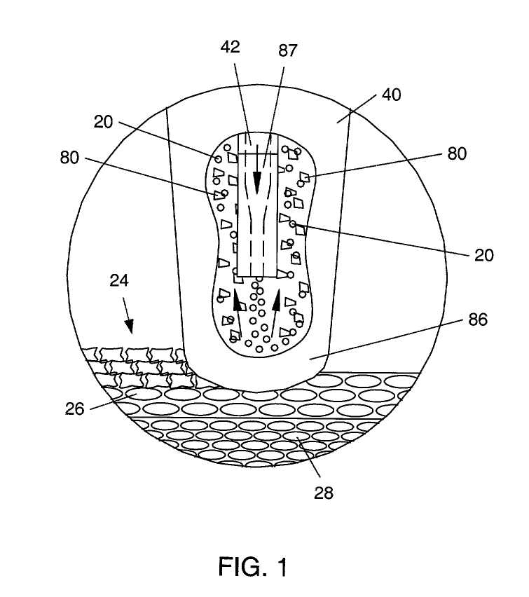

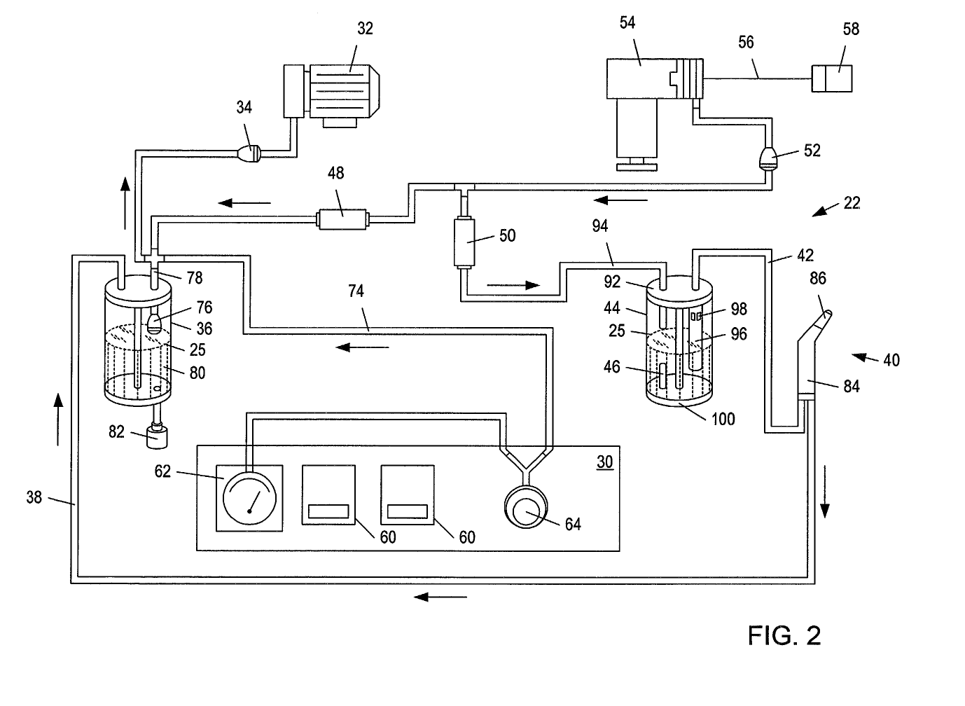

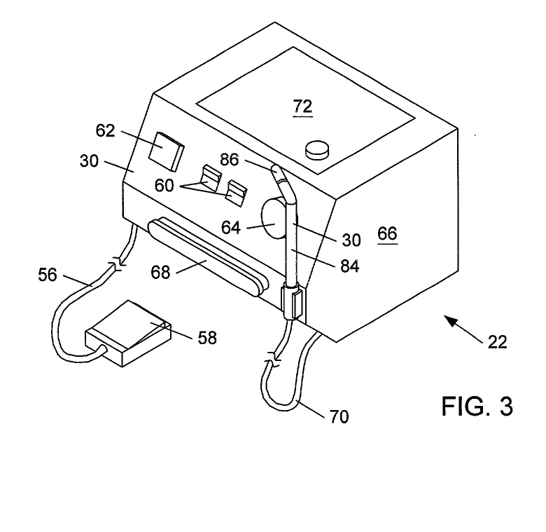

With reference to the drawings, and particularly to FIG. 1, rounded particles are designated generally as 20. Rounded particles 20 may be used in a microdermabrasion machine 22. Embodiments of microdermabrasion machines are shown in FIGS. 2 and 3. A microdermabrasion machine 22 may propel rounded particles 20 against skin 24 (shown in FIG. 1). The rounded particles 20 may be all or a portion of abrasive 25 (shown in FIG. 2 and FIGS. 5-7) used to abrade the skin 24. Embodiments of microdermabrasion machines 22 may be configured to propel the abrasive 25 against skin 24 using only vacuum, using only compressed gas, or using a compressed gas and vacuum combination. The compressed gas may be, but is not limited to, compressed air or nitrogen. When a combination of compressed gas and vacuum is used to propel abrasive 25, the compressed gas may propel the abrasive against the skin 24 within a treatment area, and the vacuum may draw the abrasive and abraded skin away from the treatment area. In certain embodiments, the abrasive 25 includes only round particles 20 with substantially smooth edges. In other embodiments, the abrasive 25 includes a mixture of round particles 20 with other types of particles. The other types of particles may include, but are not limited to sharp-edged particles, bleaching agents, drying agents, and unguents.

A microdermabrasion procedure may be used to freshen or tone skin 24, to treat wrinkles, such as aging wrinkles, to treat stretch marks, and/or to treat skin blemishes. A microdermabrasion procedure may be used to treat skin blemishes that include, but are not limited to certain forms of keratoses, acne, acne scarring, scar tissue, calluses, melasma, hyper-pigmentation, photo-damaged or sun-damaged skin, and tattoos.

The rounded particles 20 may be made of various materials including, but not limited to glass, alumina, other fired or unfired ceramic materials, and polymers. The rounded particles 20 may be solid, or the rounded particles may be hollow. The rounded particles 20 may be, but are not limited to, spheroidal, substantially ellipsoidal, substantially ovate, and/or substantially cylindrical shapes. The rounded particles 20 may have substantially smooth outer surfaces, or the rounded particles may have an overall round shape with sharp-edged surfaces. Spheroidal particles may be produced by heating the material to a liquid state. The liquid may be blown into a gas stream. Spheroidal particles may form in the gas stream. The gas stream may be cooled to form solid spheroidal particles.

A wide range of effective particle diameter sizes may be used in a microdermabrasion machine 22. A rounded particle diameter size distribution range may be from about 25 microns to about 325 microns, from about 50 microns to about 250 microns, or from about 100 microns to about 200 microns. A narrow particle diameter size distribution range may be preferred because a narrow size distribution range of particles may produce a more uniform abrasive effect within a treatment area than will a broad size distribution of particles. Also, rounded particles 20 may be commercial available in narrow size distribution ranges. For example, a microdermabrasion machine 22 may use glass beads, such as type AE Ballotini Impact Beads, which are sold by Potters Industries Inc. of Valley Forge, Pa. These glass beads have a particle diameter size distribution range of from about 90 microns to about 150 microns. Other particle diameter size ranges of rounded particles 20 may be used in a microdermabrasion machine 22. A rounded particle diameter size distribution range may be established by sieving the particles through mesh screens of selected sizes. The largest size of mesh screen through which the particles will not pass may define the lower particle diameter size limit, and the smallest size of a mesh screen through which the particles will pass may define the upper particle diameter size limit.

Rounded particles 20 that are to be used in a microdermabrasion procedure may be sterilized. The rounded particles 20 may be sterilized by any suitable procedure including, but not limited to, heat treatment, radiation treatment or chemical treatment.

Rounded particles 20 may be coated with a coloring agent. Different color coatings of rounded particles 20 may be used to distinguish between rounded particles having different characteristics. For example, blue coated rounded particles may be small particles, such as particles having a size distribution from about 45 microns to about 90 microns, while red coated rounded particles may be larger size particles, such as particles having a size distribution from about 210 microns to about 300 microns. Different color coatings may also be used to indicate that the rounded particles 20 are mixed with other types of materials such as additional abrasives (e.g., sand or aluminum oxide), bleaching agents, or drying agents.

In certain embodiments, rounded particles 20 may be mixed with other abrasive particles. For example, the rounded particles 20 may be mixed with irregularly shaped, sharp-edged aluminum oxide particles or sand particles (silicon dioxide). The rounded particles 20 may also be mixed with other types of materials. For example, the rounded particles 20 may be mixed with micro-beads of lotion or antibacterial agent. In certain embodiments, a portion of the rounded particles 20 may be coated with a material. For example, a portion of the rounded particles 20 may be coated with a lubricity agent, a lotion and/or an antibacterial agent. A lubricity agent may be added to the rounded particles 20 to reduce the abrasive effect of the particles. Other materials that may be mixed with or coated on the rounded particles 20 may include, but are not limited to coloring agents, vitamins (such as B complex vitamins and vitamin E), bleaching agents, drying agents, and unguents.

A microdermabrasion machine 22 may be used to propel rounded particles 20 against a treatment area on a patient's skin 24. Rounded particles 20 may be used to abrade portions of skin 24 and/or remove hair. A microdermabrasion machine 22 may be used to remove a portion of the epidermal skin layer 26 (shown in FIG. 1), such as the stratum corneum layer. A microdermabrasion machine 22 that uses only a vacuum to propel the rounded particles 20 may be well suited to removing only a portion of the epidermal skin layer 26. A microdermabrasion machine 22 that uses only a vacuum to propel the rounded particles 20 may be used to freshen or tone the skin 24.

A microdermabrasion machine 22 may be used to remove the entire epidermal skin layer 26. A microdermabrasion machine 22 may also be used to remove the epidermal layer 26 and a portion of dermal skin layer 28. The dermal skin layer 28 is shown in FIG. 1. A microdermabrasion machine 22 that uses a compressed gas and a vacuum to propel rounded particles 20 may be used to remove the epidermal layer skin layer 26 and a portion of the dermal skin layer 28. A microdermabrasion machine 22 that uses a compressed gas and a vacuum to propel rounded particles 20 may be used to treat skin blemishes, tattoos and the like within the dermal skin layer 28. A microdermabrasion machine 22 may be used in conjunction with other treatment procedures to treat skin blemishes, tattoos and the like.

A microdermabrasion machine 22 may be obtained from Aesthetic Technologies, Inc. of Broomfield, Colo. For example, the Espirit™ Model 1500 and the Prestige™ Model 2500 machines may be obtained from Aesthetic Technologies and used as is described herein. FIG. 2 shows a representation of an embodiment of a microdermabrasion machine 22. The machine 22 may include instrument panel 30, vacuum pump 32, vacuum pump filter 34, waste receptacle 36, waste line 38, handpiece 40, supply line 42, and supply receptacle 44. The machine 22 may optionally include heater 46, solenoid valve 48, solenoid valve 50, filter 52, compressed gas source 54, control line 56, and foot control 58. The instrument panel 30 may include switches 60, gauge 62, and control 64.

FIG. 3 shows a perspective representation of an embodiment of a microdermabrasion machine 22. The machine 22 may include housing 66, handle 68, tubing 70, and door 72 in addition to the instrument panel 30, the handpiece 40 and the optional control line 56 and foot control 58. The handle 68 of the machine 22 may allow the machine to be easily positioned at a desired location when the machine is placed on a wheeled instrument carrier. The tubing 70 may include a portion of the waste line 38 and a portion of the supply line 42. The door 72 may provide access to the working parts of the machine 22, including the waste receptacle 36 and the supply receptacle 44.

A vacuum pump 32 (shown in FIG. 2) may be used to draw rounded particles 20 from a supply receptacle 44 to a waste receptacle 36. The vacuum produced by the vacuum pump 32 may be set to a desired level by adjusting control 64 to increase or decrease the amount of air drawn to the vacuum pump through line 74. The vacuum produced by the vacuum pump 32 during a microdermabrasion procedure may be between about 10 centimeters of mercury and about 50 centimeters of mercury. Filter 34 may inhibit small particles from reaching the vacuum pump 32. The vacuum pump 32 preferably includes internal filters that inhibit small particles from reaching the seals and moving parts of the vacuum pump.

A waste receptacle 36 may include filter 76 at an end of line 78. The filter 76 may inhibit used rounded particles 20 and abraded waste material 80 (shown in FIG. 1 and FIGS. 5-7) from being drawn to the vacuum pump 32. Waste line 38 may enter into the waste receptacle 36. The waste line 38 conveys used rounded particles 20 and abraded waste material 80 to the waste receptacle 36. The waste receptacle 36 may include optional drain 82. The drain 82 allows used abrasive particles and abraded waste material 80 to be removed from the waste receptacle 36 when the drain is opened. Alternatively, the waste receptacle 36 may be a disposable unit that is replaceable after each use or after a certain number of uses.

If the microdermabrasion machine 22 includes a compressed gas source 54, solenoid valve 48 may be periodically cycled. Cycling the solenoid valve 48 may allow a charge of compressed gas to be directed through line 78 to the filter 76. The charge of compressed gas may clear the filter 76. Solenoid valve 48 may be automatically activated at periodic intervals during a microdermabrasion procedure, or the solenoid valve may be manually activated by a user of the microdermabrasion machine 22. In embodiments of microdermabrasion machines 22 that do not include compressed gas sources 54 for propelling rounded particles 20, a separate gas source may be coupled to line 78 so that a charge of gas may be directed to the filter 76.

A handpiece 40 of a microdermabrasion machine 22 may be used to direct rounded particles 20 against skin 24 in a treatment area. The handpiece 40 may include handle 84 and tip 86. A user may grip the handle 84 and guide the tip 86 of the handpiece 40 over skin 24 within a treatment area. The tip 86 of the handpiece 40 may be canted at an angle relative to the handle 84 of the handpiece. The tip 86 of the handpiece 40 may include a visual window that allows the operator of the machine to see the treatment area during a microdermabrasion procedure. The tip 86 of the handpiece 40 may be a disposable unit that can be replaced after each use. The handpiece 40 may include a safety mechanism that inhibits rounded particles 20 from traveling through the handpiece when the tip 86 of the handpiece is not in contact with a surface, such as a treatment area. The handpiece 40 may be attached to the housing 66 of the machine by tubing 70.

FIG. 1 shows a cut-away view of a tip 86 of a handpiece 40 that is in contact with skin 24. Supply line 42 stops within the handpiece 40 a distance above the skin 24. Rounded particles 20 from supply receptacle 44 are directed through the supply line 42 and against the skin 24 during a microdermabrasion procedure. Vacuum produced by vacuum pump 32 may draw air and abrasive 25 from the supply container 44, through the supply line 42, to nozzle 87. The nozzle 87 directs the abrasive 25 against the skin 24 through an opening in tip 86 of the handpiece 40. The used rounded particles 20 and abraded material 80 may be drawn to waste line 38 through an annular space between inner surface of the handpiece 40 and the outer surface of the supply line 42. As shown schematically in FIG. 2, abrasive 25 may be directed from the handpiece 40 to the waste receptacle 36 through the waste line 38. Arrows shown within the handpiece 40 in FIG. 2 indicate flow direction of gas and/or particles during the microdermabrasion procedure.

A supply receptacle 44 (shown in FIG. 2) may include lid 92, optional heater 46, inlet line 94 and tube 96. In an embodiment, lid 92 of the supply receptacle 44 is removable. Rounded particles 20 and/or abrasive 25 may be poured into the supply receptacle 44 from a bulk supply container. In other embodiments, top of the supply receptacle 44 may include a removable cover over an opening. Additional abrasive 25 may be placed in the supply receptacle 44 through the opening. Alternatively, the supply receptacle 44 may be a prepackaged canister that has a sufficient supply of abrasive 25 to perform a microdermabrasion procedure or a limited number of microdermabrasion procedures. A heater 46 may be used to keep the abrasive particles 25 dry. Dry particles may be less likely to clump than damp particles. The particles 25 may become damp due to contact with moisture in gas that flows into the supply receptacle 44 through the inlet line 94.

Other materials may be mixed with rounded particles 20 and stored in supply receptacle 44. Such other materials may include, but are not limited to other abrasive particles (e.g, sand or aluminum dioxide), lotions, or antibacterial agents. When rounded particles 20 are transported from the supply receptacle 44 and propelled against skin 24 during a microdermabrasion procedure, the other materials mixed with the rounded particles may also be propelled against the skin. Alternatively, the other materials may be coupled to a handpiece 40 by separate transport systems. The separate transport systems may propel the other materials against the skin 24 during a microdermabrasion procedure. The separate transport systems may use supply line 42 and/or waste line 38. Alternatively, the separate transport systems may be independent systems.

A tube 96 and supply line 42 may work together to allow rounded particles to be drawn out of the supply receptacle 44 through the supply line. The tube 96 may have openings 98 near a top of the tube. The supply line 42 may be inserted through lid 92 of the supply receptacle 44 into the tube 96. A gap should be left between an end of the supply line 42 and bottom 100 of the supply receptacle 44. When a vacuum is produced by vacuum pump 32 or when a compressed gas flows through inlet line 94 and into the supply receptacle 44, gas may be directed through the openings 98 in the tube 96 into an annular space between the tube and the supply line 42. The gas may flow into the supply line 42 towards handpiece 40. A portion of the rounded particles within the supply receptacle 44 may be carried by the gas towards the handpiece 40.

A microdermabrasion machine 22 may include compressed gas source 54 and solenoid valve 50. The compressed gas source 54 may be a compressor that supplies compressed air to supply receptacle 44. Alternatively, the compressed gas source 54 may be a compressed gas cylinder. A microdermabrasion machine 22 that includes a compressed gas source 54 may be able to cause more abrasion of skin 24 in a treatment area than can be produced by a microdermabrasion machine that does not include a compressed gas source. A user of the microdermabrasion machine 22 may control the driving force imparted to rounded particles 20 by the compressed gas with a foot control 58. The compressed gas source 54 or a control mechanism coupled to the compressed gas source may be operatively connected to the foot control 58 by cable 56. The pressure of compressed gas supplied to drive the rounded particles 20 may range from a little above 0 psi to about 45 psi. Typically, the compressed gas pressure during a microdermabrasion procedure would be less than about 30 psi. A filter 52 may be located between the compressed gas source 54 and the supply receptacle 44 to inhibit the introduction of contaminants into the microdermabrasion machine 22.

Solenoid valve 50 may allow a microdermabrasion machine 22 that is equipped with a compressed gas source 54 to propel rounded particles 20 with vacuum produced by the vacuum pump 32, or with compressed gas from the compressed gas source and with vacuum produced by the vacuum pump. When the solenoid valve 50 is in a first position, an input side of the solenoid valve 50 may be open to the atmosphere so that air may be drawn through inlet line 94 to supply receptacle 44 during a microdermabrasion procedure. When the solenoid valve 50 is in the first position, the microdermabrasion machine 22 may be configured to use only vacuum to propel the rounded particles 20. When the solenoid valve 50 is in a second position, gas from the compressed gas source 54 may flow through inlet line 94 to the supply receptacle 44. When the solenoid value 50 is in the second position, the rounded particles may be propelled by both the compressed gas and vacuum produced by the vacuum pump 32.

To use a microdermabrasion machine 22 to perform a microdermabrasion procedure, an operator may visually check that supply receptacle 44 has a supply of rounded particles 20 and that waste receptacle 36 does not need to be emptied. The supply receptacle 44 may contain other material, such as sand, or irregularly shaped aluminum oxide particles, in addition to rounded particles 20.

After confirming that supply receptacle 44 has rounded particles 20 and that waste receptacle 36 has room to hold used abrasive particles and abraded material 80, an operator may place tip 86 of handpiece 40 against a treatment area and turn on microdermabrasion machine 22. The operator may adjust control 64 so that vacuum pump 32 produces a desired amount of vacuum. When the vacuum pump 32 produces a vacuum, abrasive particles 25 may be drawn from the supply receptacle 44, through the handpiece 40, and into contact with skin 24 beneath the tip 86. If the tip 86 of handpiece 40 is not placed against a surface, such as skin 24 of a treatment area, air drawn into the tip by the vacuum pump 32 may inhibit abrasive particles from being drawn from the supply receptacle 44. If compressed gas is used to propel rounded particles 20 in addition to the vacuum produced by the vacuum pump 32, the operator may use foot control 58 to control how much compressed gas is used to propel the rounded particles.

An operator of microdermabrasion machine 22 may move handpiece tip 86 over skin 24 within a treatment area. Rounded particles 20 may abrade and remove portions of the skin 24. The used rounded particles and removed material 80 may be drawn through waste line 38 by vacuum pump 32. The vacuum pump 32 may remove substantially all of the rounded particles 20 and removed material 80 from the skin 24.

Rounded particles 20 used during a microdermabrasion procedure may include glass beads. During a test of the effectiveness of glass beads as opposed to irregularly shaped aluminum oxide particles as an abrasive, glass beads and irregularly shaped aluminum oxide particles were directed against pages of a telephone book from the same microdermabrasion machine. When the abrasives were propelled by compressed air and removed by a vacuum, the glass beads were observed to be less effective than the aluminum oxide particles at abrading pages of the phone book. When the abrasives were propelled only by vacuum, the glass beads produced significantly less effect than did the irregularly shaped aluminum oxide particles. The glass beads were estimated to be approximately one third as effective as the irregularly shaped aluminum oxide particles when only a vacuum was used to propel the abrasive particles.

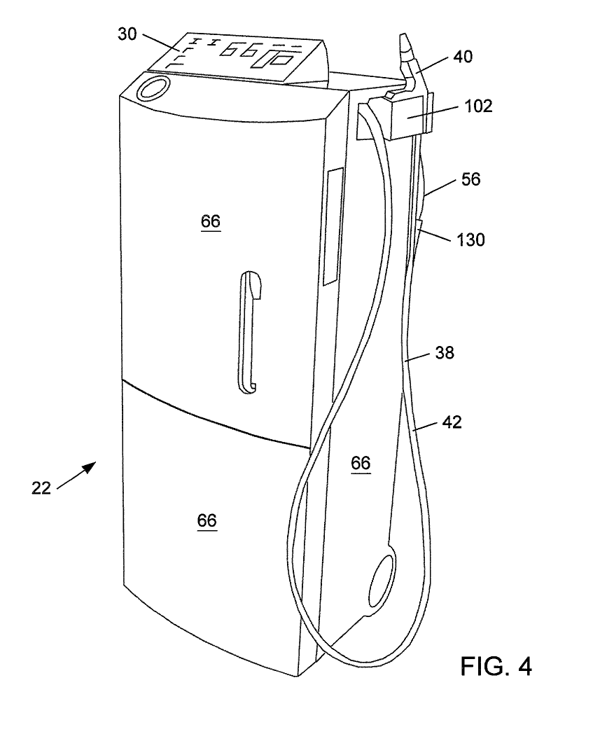

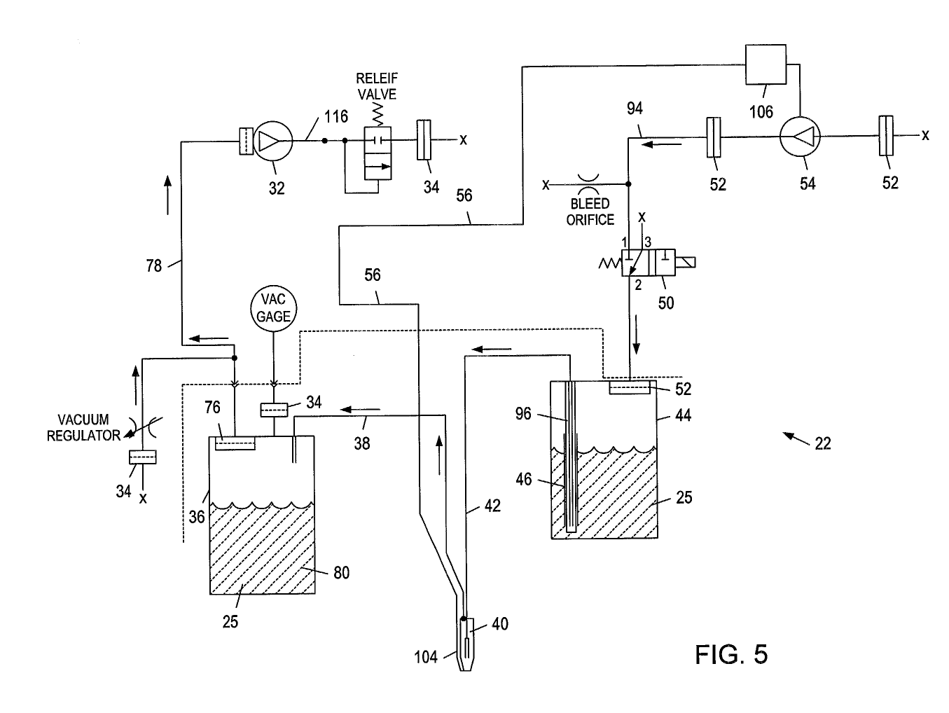

FIG. 4 shows an alternate embodiment of a microdermabrasion machine 22 that includes housing 66, instrument panel 30, supply line 42, waste line 38, control line 56, handpiece 40, and handpiece holder 102. The control line 56 may not be needed if the handpiece 40 does not include an activation mechanism that controls propulsion of abrasive 25 through the handpiece. The housing 66 may include an interior compartment that holds components of the microdermabrasion machine 22. As shown schematically in FIGS. 5, 6, and 7 the microdermabrasion machine 22 may include vacuum pump 32, filters 34, 52, 76, supply container 44, waste container 36, pick-up tube 96, heater 46, and compressor 54.

A handpiece 40 may be placed in the handpiece holder 102 when not in use. An electrical circuit, or other type of circuit, may be coupled to the handpiece 40 so that the vacuum pump 32 turns on when the handpiece is removed from the holder 102 and so that the vacuum pump turns off when the handpiece is placed in the holder.

FIG. 5 shows an embodiment of a microdermabrasion machine 22 that includes a compressor 54 that is electrically controlled by activation of handpiece 40. Control line 56 is an electrical connection between activation mechanism 104 in handpiece 40 and transducer 106. Transducer 106 may be a switch. When the activation mechanism 104 is engaged, the transducer 106 may send a signal to the compressor 54 that turns the compressor on. When the activation mechanism 104 is disengaged, the transducer 106 may send a signal to the compressor 54 that turns the compressor off.

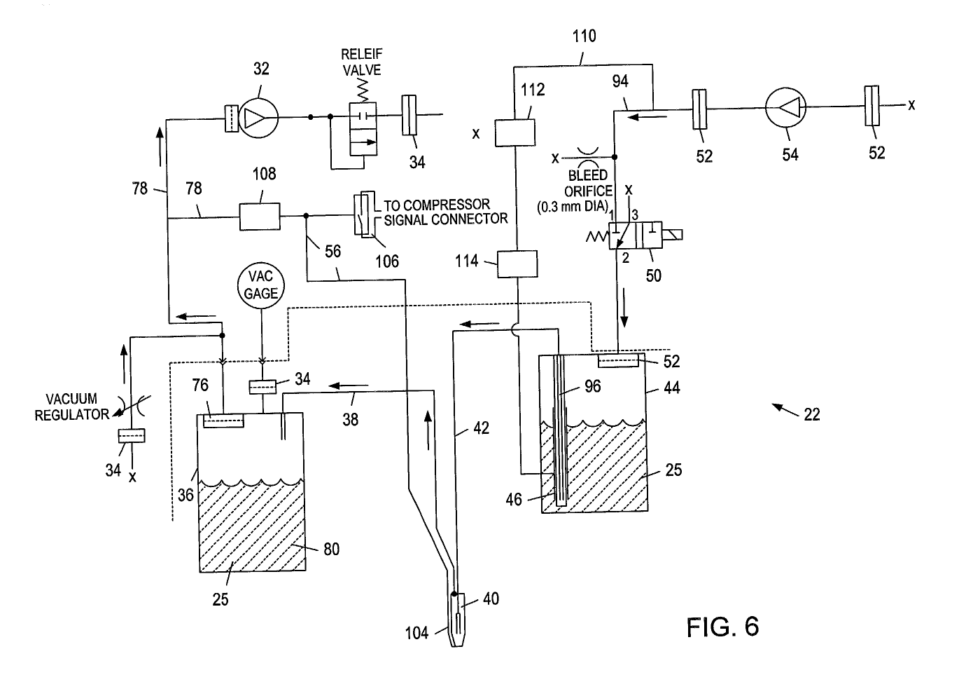

FIG. 6 shows an embodiment of a microdermabrasion machine 22 that includes a compressor 54 that is pneumatically controlled by activation of handpiece 40. Control line 56 is a pneumatic line that draws a vacuum through activation mechanisms 104. When the activation mechanism 104 is engaged, the transducer 106 registers an increase in vacuum. The transducer 106 may than send a signal to the compressor 54 that turns the compressor on. When the activation mechanism is disengaged, the transducer registers a decrease in vacuum. The transducer 106 may then send a signal to the compressor 54 that turns the compressor off. Vacuum reducer 108 may be placed in the control line 56 to maintain the vacuum pulled through the line at a level within the working range of the transducer 106.

As shown in the microdermabrasion machine embodiment of FIG. 6, a microdermabrasion machine 22 may include line 110 that supplies air or gas through solenoid 112 to supply receptacle 44. The solenoid 112 may periodically cycle to direct a charge of air or gas into the supply receptacle 44 adjacent to tube 96. Drawing abrasive 25 through the tube 96 may cause the formation of a cone shaped void within the abrasive adjacent to the tube. A periodic charge of air or gas into the supply receptacle 44 may disrupt formation of the cone adjacent to the tube 96. Line 110 may supply air from the discharge side of the vacuum pump 32, from compressor 54 (as shown in FIG. 6) or from a separate compressor coupled to the microdermabrasion machine 22. Pressure controller 114 may control the pressure applied to the supply receptacle 44. In an alternate embodiment, a solenoid and a control valve are coupled to a vacuum line, which is coupled to inlet line 94. The solenoid may be periodically cycled so that a vacuum is drawn within the supply receptacle 44. The control valve allows gas supply from the compressor or atmosphere to be bypassed during a time when vacuum is being drawn within the supply receptacle. The vacuum may disrupt the formation of the cone adjacent to the tube 96.

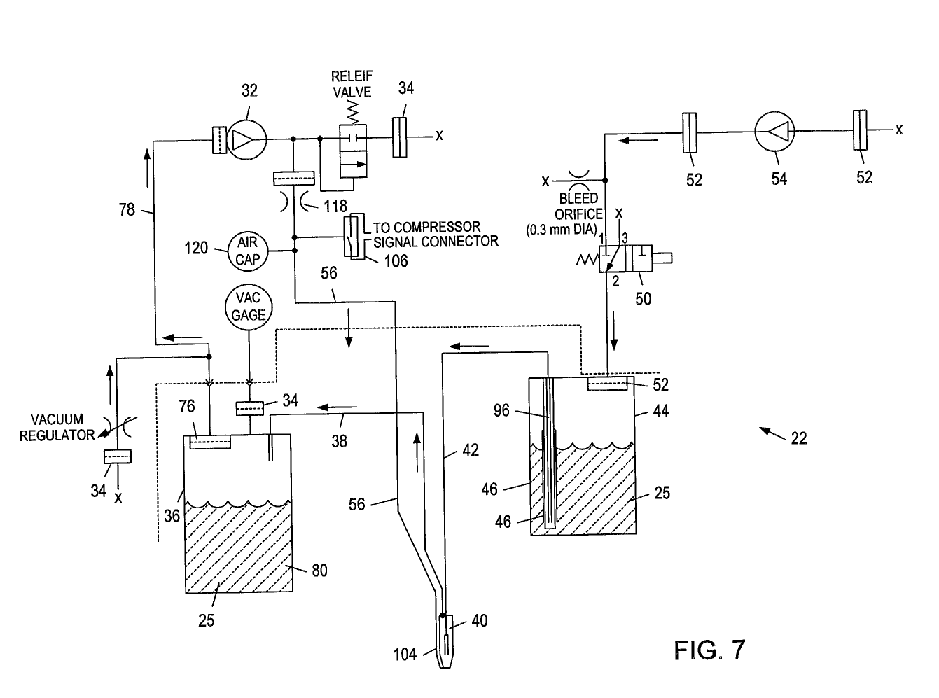

FIG. 7 shows an embodiment of a microdermabrasion machine 22 that includes a compressor 54 that is pneumatically controlled by activation of handpiece 40. Control line 56 may be a pneumatic line that applies air flow through activation mechanisms 104. A separate compressor may be coupled to the system to provide a low pressure flow to the control line 56. Alternately, a low pressure flow may be supplied to the control line 56 from exhaust line 116 of the vacuum pump 32. Orifice 118 may be coupled to the control line 56. The orifice 118 may reduce the consumption of pressurized air from the control line 56, and the orifice may reduce the pressure of the exhaust line to near atmospheric pressure. Air capacitor 120 may also be coupled to the control line 56. The air capacitor 120 may make the microdermabrasion machine 22 resistant to short term pressure fluctuations that occur when moving the handpiece 40 over rough or irregular skin. When the activation mechanism 104 is engaged, the transducer 106 registers an increase in pressure. The transducer 106 may than send a signal to the compressor 54 that turns the compressor on. When the activation mechanism is disengaged, the transducer registers a decrease in pressure. The transducer 106 may then send a signal to the compressor 54 that turns the compressor off.

If more abrasion is needed than can be provided by using only the vacuum pump 32 to drive the abrasive 25, an operator of the microdermabrasion machine 22 may engage a system that drives the abrasive with a compressed gas. Compressor 54 may supply the compressed gas. The compressed gas may be air. Alternately, an external gas supply may be coupled to gas supply line 94. The external gas supply may be a compressed gas cylinder, such as a nitrogen cylinder or an air cylinder, which is coupled to a pressure regulator. The system may be engaged by activation mechanism 104 in the handpiece 40. Alternately, the system may be engaged by another type of mechanism, such as a foot pedal 58 (shown in FIG. 3). The activation mechanism 104 may be an electrically operated mechanism or a pneumatically operated mechanism. The activation mechanism 104 may include both an electrically operated mechanism and a pneumatically operated mechanism.

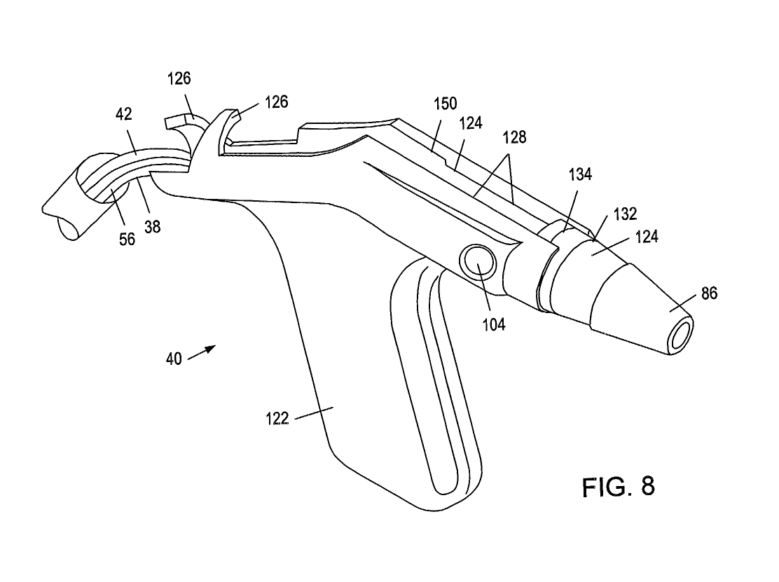

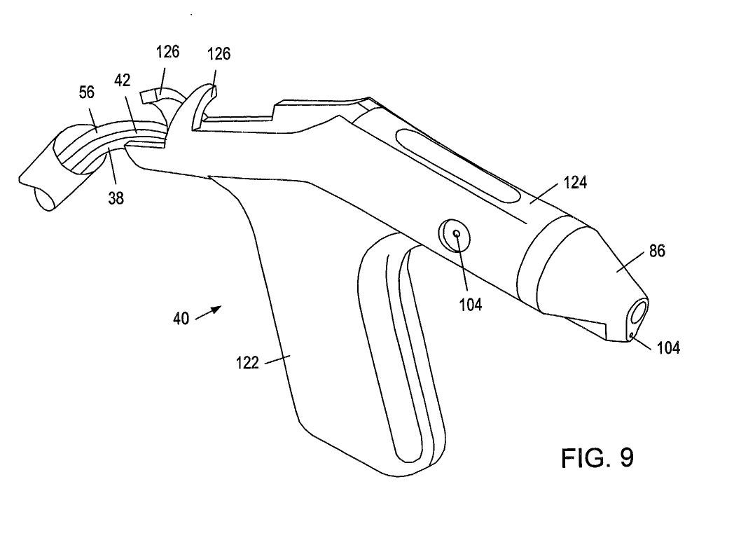

FIGS. 8 and 9 show embodiments of assembled handpieces 40. The handpiece 40 may include grip 122 and body 124. The grip 122 may be a plastic handle that is formed in two pieces. The two pieces may snap together around the body 124. The grip 122 may include tubing tabs 126 that engage and keep supply line 42 and waste line 38 out of an operator's way. The grip 122 may resemble a pistol grip. The grip 122 may provide a comfortable structure for an operator to hold during a microdermabrasion procedure. The grip 122 may be ergonomically shaped so that the grip promotes proper positioning of an operator's arm and hand during use. The grip 122 may orient the body 124 at a convenient angle for contact with skin 24 of a patient.

FIG. 8 shows an embodiment of a handpiece 40 that has an electrically operated activation mechanism 104 for controlling compressor 54. FIG. 5 shows a schematic diagram of a microdermabrasion machine 22 that may use the type of handpiece 40 shown in FIG. 8. The control line 56 for an electrically operated mechanism may be a flexible tube that encloses a pair of wires. The wires may be coupled to electrical contacts 128 on the body 124. Pressing activation mechanism 104 may cause contact against the contacts 128 to complete an electrical circuit that engages the compressor 54. Quick connect 130 (shown in FIG. 4) may allow the control line 56 to be disconnected so that the handpiece 40 can be cleaned, maintained, or replaced. As shown in FIG. 8, the grip 122 may also include recess 132. The recess 132 may engage body protrusion 134 to couple the body 124 to the grip 122.

FIG. 5 depicts a schematic representation of a microdermabrasion machine 22 that includes a handpiece 40 that uses an electrically operated activation mechanism 104 to engage compressor 54, such as the handpiece shown in FIG. 8. To engage the compressor 54, an operator pushes activation mechanism 104 with a finger. Pushing the activation mechanism 104 may compress a spring and complete an electrical circuit with contacts 128 that causes the compressor 54 to turn on. Releasing the activation mechanism 104 may allow the spring to return to an initial position, break the electrical circuit, and cause the compressor 54 to turn off.

FIG. 6 and 7 depict schematic representations of microdermabrasion machines 22 that may use the handpiece 40 shown in FIG. 9. FIG. 9 shows an embodiment of a handpiece 40 that has two pneumatically engaged activation mechanisms 104 that activate compressor 54. Other embodiments may have only a single pneumatically operated mechanism 104, both pneumatic and electrical activation mechanisms, no activation mechanisms, or more than two activation mechanisms. The handpiece 40 may include grip 122 and body 124. The grip 122 may be a plastic handle that is formed in two pieces. The pieces may snap together. The grip 122 may include tabs 126 that engage and keep supply line 42, waste line 38 and control line 56 out of an operator's way. The control line 56 may be tubing. Covering both the activation mechanism 104 on the tip 86 and the activation mechanism on the side of the body 124 may be required to engage the compressor 54.

A body 124 of a handpiece 40 may be formed of a number of pieces. FIG. 10 shows an exploded view of a body 124 for a pneumatic controlled handpiece 40. In an embodiment, the pieces include boot 136, tube adapter 138, tip body 140, nozzle 87, and tip 86. The pieces of the body 124, or selected pieces of the body, may be reusable. Alternately, pieces of the body 124, or selected pieces of the body, may be disposable after a single use or after a number of uses. Similarly, the grip 122 may be made as a disposable or a reusable component. The grip 122, and the pieces of the body 124 may be formed, but are not limited to being formed, by molding and/or casting. A reusable body or tip may be made of polyesterimide, polysulfone or other resistant polymer so that the pieces may be sterilized within an autoclave or by chemical sterilization. A disposable body 124 may be made of acrylonitrile-butadiene-styrene (ABS) co-polymer or other suitable polymer.

In an embodiment, separate pieces that form the body 124 may be formed by injection molding. Disposable pieces may be formed of a plastic material such as ABS co-polymer. Reusable pieces may be formed of a plastic material such as polysulfone or polyesterimide. The polymers used to form disposable and reusable components may have approximately the same expansion properties so that the same molds may be used to form disposable pieces as well as reusable pieces.

Some pieces of a body 124 may be removably coupled together. For example, the tip 86 may be removably coupled to the tip body 140. Removably coupled pieces may be coupled together by friction locking, protrusion and groove engagement, or by another type of coupling system. Other pieces may be non-removably coupled together. For example, the tube adapter 138 may be non-removably coupled to the boot 136. Non-removably coupled pieces may be coupled together by glue, sonic welding, or other type of permanent coupling system.

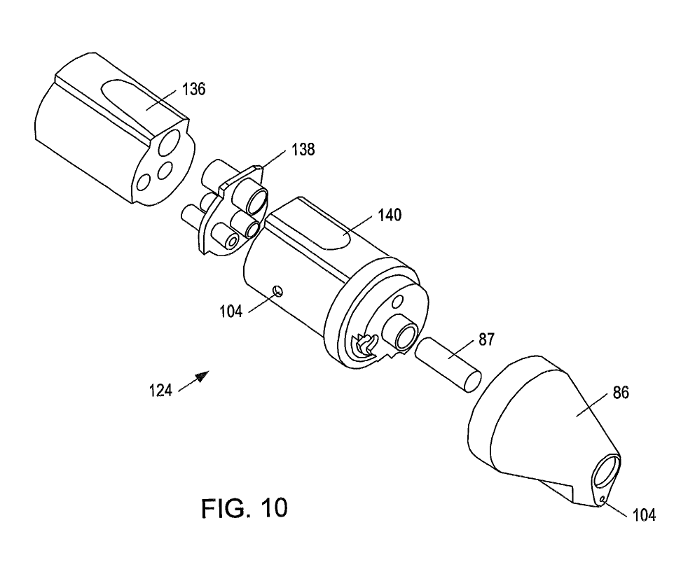

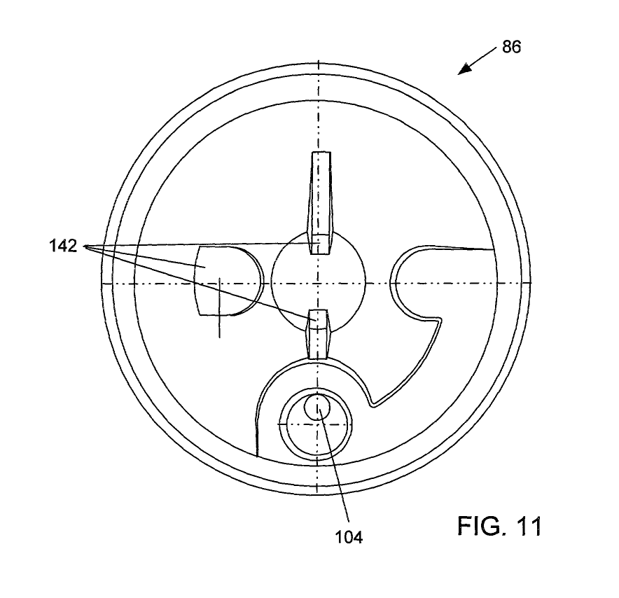

A boot 136 may allow supply line 42, waste line 38, and control line 56 to be easily connectable to the body 124. The boot 136 may be a reusable component, or the boot may be disposable. The tube adapter 138 may couple the boot 136 to the tip body 140. The tip body 140 may be coupled to the tube adapter 138. The nozzle 87 may be press fit into the tip body 140. The tip 86 may be coupled to the tip body 140. The tip 86 may include structural members 142, which are shown in FIG. 11, that support the nozzle 87 when the nozzle is coupled to the tip body 140.

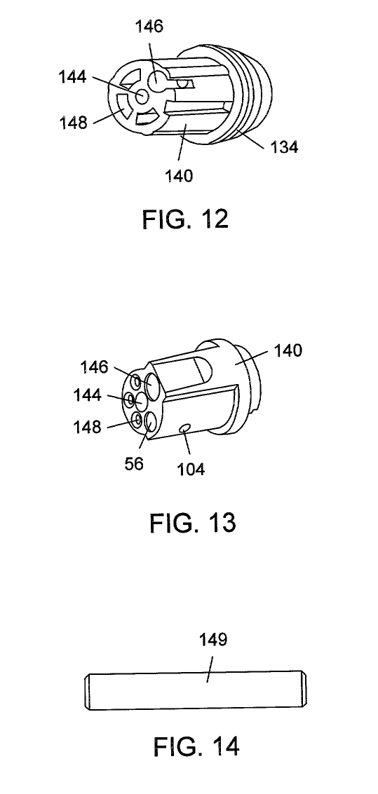

The tip body 140 may include feed tube 144 and suction tube 146, as shown in FIGS. 12 and 13. The feed tube 144 may couple to the supply line 42 and to the nozzle 87. The suction tube 146 may couple to the waste line 38. The body 124 may also include cores 148. The cores 148 may reduce the amount of plastic needed to form the body 124 and inhibit warpage of the body during formation. A metallic insert tube 149, which is shown in FIG. 14, may be positioned within the feed tube 144. An insert tube 149 may also be positioned within suction tube 146. The insert tubes 149 may reduce the abrasion of the body 124 during a microdermabrasion procedure. The insert tubes 149 may be made of brass or another type of metal. An insert tube 149 positioned within the feed tube 144 may not have the same length or diameter as an insert tube positioned within the suction tube 146.

The body 124 may include a nozzle 87. A disposable nozzle 87 may be made of an abrasion resistant polymer, a cast metal or a plastic with a metal insert. A disposable nozzle 87 should be designed so that the nozzle will abrade sufficiently to noticeably impede performance after one use. The noticeable impairment of performance may force a user to replace a disposable nozzle 87 after each use. A reusable nozzle 87 may be made of tungsten carbide. A tungsten carbide nozzle 87 may last for numerous treatments.

In an embodiment of a handpiece 40, body 124 may also include a contact sensor that registers when tip 86 of the handpiece is placed against a surface, such as patient's skin 24. When the tip 86 is placed against a surface, contact 150 (shown in FIG. 8) at an end of the body 124 may complete an electrical circuit. The completion of the circuit and engagement of the activation mechanism 104 by the operator may both be required to engage the compressor 54. In alternate embodiments, contact sensors that register when tips 86 of handpieces 40 are placed against surfaces may be pneumatic sensors, such as activation mechanism 104 in tip 86 of the handpiece embodiment shown in FIG. 9.

A feed tube 144 and a suction tube 146 of a handpiece 40 may have small volumes. The small volumes may allow the handpiece 40 to quickly begin to abrade skin 24 when the handpiece 40 or is positioned against the skin and when the microdermabrasion machine 22 is turned on. The small volumes may also allow the vacuum pump 32 to completely empty the tubes 144, 146 when the handpiece 40 is removed from contacting the skin 24. Having the vacuum pump 32 empty the tubes 144, 146 may inhibit abrasive 25 and abraded matter 80 from falling out of the handpiece 40 when the handpiece is removed from contact with the skin 24.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A system for treating skin, comprising:
an abrasive comprising rounded particles; and
a machine configured to propel the abrasive against the skin during use.

2. The system of claim 1, wherein the rounded particles are sterilized.

3. The system of claim 1, further comprising a coating on the rounded particles.

4. The system of claim 3, wherein the coating comprises a lubricity agent.

5. The system of claim 3, wherein the coating comprises a coloring agent.

6. The system of claim 3, wherein the coating comprises a bleaching agent.

7. The system of claim 3, wherein the coating comprises a drying agent.

8. The system of claim 3, wherein the coating comprises a vitamin.

9. The system of claim 3, wherein the coating comprises a lotion.

10. The system of claim 3, wherein the coating comprises an antibacterial agent.

11. The system of claim 1, wherein the abrasive is used to treat stretch marks.

12. The system of claim 1, wherein the abrasive is used to treat acne scarring.

13. The system of claim 1, wherein the abrasive is used to treat wrinkles.

14. The system of claim 1, wherein the rounded particles are hollow.

15. The system of claim 1, wherein the rounded particles have a size range distribution between about 25 microns and about 325 microns.

16. The system of claim 1, wherein the rounded particles have a size range distribution from about 45 microns to about 90 microns.

17. The system of claim 1, wherein the rounded particles have a size range distribution from about 90 microns to about 150 microns.

18. The system of claim 1, wherein the rounded particles comprise ceramic material.

19. The system of claim 1, wherein the rounded particles comprise glass beads.

20. The system of claim 1, wherein the rounded particles comprise aluminum oxide.

21. The system of claim 1, wherein the abrasive further comprises irregularly shaped aluminum oxide particles.

22. The system of claim 1, wherein the abrasive further comprises sand.

23. The system of claim 1, wherein the rounded particles comprise spheroidal shaped particles.

24. The system of claim 1, wherein the rounded particles comprise substantially ellipsoidal shaped particles.

25. The system of claim 1, wherein the rounded particles comprise substantially cylindrical shaped particles.

26. The system of claim 1, wherein the machine further comprises a vacuum pump, and wherein the vacuum pump produces a vacuum that propels the abrasive against the skin.

27. The system of claim 1, further comprising a compressed gas line coupled to the machine, wherein compressed gas within the compressed gas line propels the abrasive against the skin during use.

28. The system of claim 1, wherein the machine further comprises a compressor, and wherein the compressor is configured to produce compressed air that propels the abrasive against the skin during use.

29. The system of claim 1, wherein the rounded particles abrade a portion of the skin during use.

30. The system of claim 1, further comprising an antibacterial agent, and wherein the machine is configured to propel the antibacterial agent against the skin during use.

31. The system of claim 1, further comprising a lotion, and wherein the machine is configured to propel the lotion against the skin during use.

32. The system of claim 1, further comprising a bleaching agent, and wherein the machine is configured to propel the bleaching agent against the skin during use.

33. The system of claim 1, further comprising a drying agent, and wherein the machine is configured to propel the drying agent against the skin during use.

34. The system of claim 1, further comprising a second abrasive, the second abrasive having a different particle size distribution than the abrasive, and wherein a color of the second abrasive differs from a color of the first abrasive.

35. A system for treating skin, comprising:
a vacuum pump;
a handpiece operatively coupled to the vacuum pump;
a storage receptacle coupled to the handpiece by a conduit;
a waste receptacle coupled to the handpiece by a conduit; and
an abrasive comprising rounded particles.

36. The system of claim 35, wherein the abrasive is storable within the storage receptacle.

37. The system of claim 35, wherein vacuum produced by the vacuum pump draws abrasive from the storage receptacle, through the handpiece, and to the waste receptacle during use, and wherein the handpiece is configured to direct abrasive against the skin during use.

38. The system of claim 35, wherein the rounded particles comprise glass beads.

39. The system of claim 35, wherein the rounded particles comprise ceramic particles.

40. The system of claim 35, wherein the abrasive further comprises irregularly shaped aluminum oxide particles.

41. The system of claim 35, wherein the abrasive further comprises sand.

42. The system of claim 35, wherein the rounded particles comprise spheroidal shaped particles.

43. The system of claim 35, wherein the rounded particles comprise substantially ellipsoidal shaped particles.

44. The system of claim 35, wherein the rounded particles comprise substantially cylindrical shaped particles.

45. The system of claim 35, further comprising a compressed gas line operatively coupled to the handpiece, wherein compressed gas within the compressed gas line propels the rounded particles against the skin during use.

46. The system of claim 35, wherein the rounded particles have sizes ranging between about 25 microns to about 325 microns.

47. The system of claim 35, further comprising an antibacterial agent, wherein the antibacterial agent is propelled against the skin during use.

48. The system of claim 35, further comprising a lotion, wherein the lotion is propelled against the skin during use.

49. The system of claim 35, wherein the rounded particles abrade the skin during use.

50. The system of claim 35, wherein the rounded particles are hollow.

51. The system of claim 35, further comprising a second abrasive, the second abrasive having a different particle size distribution than the abrasive, and wherein a color of the second abrasive differs from a color of the first abrasive.

52. A system for treating skin, comprising:
an abrasive;
a gas line;
a receptacle coupled to the gas line, the receptacle configured to store the abrasive; and
a handpiece coupled to the receptacle by a conduit.

53. The system of claim 52, wherein gas that flows through the gas line is configured to entrain a portion of abrasive stored within the receptacle, and transport the portion of abrasive to the handpiece, and wherein the handpiece is configured to direct abrasive against the skin during use.

54. The system of claim 52, further comprising a vacuum line operatively coupled to the handpiece, wherein vacuum supplied by the vacuum line is used to draw the rounded particles to a waste receptacle.

55. The system of claim 52, wherein the rounded particles are hollow.

56. The system of claim 52, wherein the rounded particles comprise glass beads.

57. The system of claim 52, wherein the rounded particles comprise spheroidal shaped particles.

58. The system of claim 52, further comprising an antibacterial agent, wherein the antibacterial agent is propelled against the skin during use.

59. The system of claim 52, further comprising a lotion, wherein the lotion is propelled against the skin during use.

60. The system of claim 52, wherein the gas comprises air.

61. An abrasive for use in a machine that propels the abrasive against skin, comprising:
rounded particles; and
a coating on the rounded particles.

62. The abrasive of claim 61, wherein the rounded particles comprise ceramic particles.

63. The abrasive of claim 61, wherein the rounded particles are hollow.

64. The abrasive of claim 61, wherein the rounded particles comprise glass beads.

65. The system of claim 61, wherein the abrasive further comprises irregularly shaped aluminum oxide particles.

66. The system of claim 61, wherein the abrasive further comprises sand.

67. The abrasive of claim 61, wherein the rounded particles comprise spheroidal shaped particles.

68. The abrasive of claim 61, wherein the rounded particles comprise substantially cylindrical shaped particles.

69. The abrasive of claim 61, wherein the rounded particles are sterilized.

70. The abrasive of claim 61, wherein the coating comprises a lubricity agent.

71. The abrasive of claim 61, wherein the coating comprises a coloring agent.

72. The abrasive of claim 61, wherein the coating comprises a bleaching agent.

73. The abrasive of claim 61, wherein the coating comprises a drying agent.

74. The abrasive of claim 61, wherein the coating comprises a vitamin.

75. The abrasive of claim 61, wherein the coating comprises a lotion.

76. The abrasive of claim 61, wherein the coating comprises an antibacterial agent.

77. The abrasive of claim 61, further comprising aluminum oxide particles mixed with the rounded particles.

78. The abrasive of claim 61, further comprising a bleaching agent mixed with the rounded particles.

79. The abrasive of claim 61, further comprising a drying agent mixed with the rounded particles.

80. The abrasive of claim 61, further comprising a vitamin mixed with the rounded particles.

81. The abrasive of claim 61, further comprising lotion mixed with the rounded particles.

82. The abrasive of claim 61, further comprising an antibacterial agent mixed with the rounded particles.

83. An abrasive for use in a machine that propels the abrasive against skin, comprising:
rounded particles mixed with sharp-edged particles.

84. The abrasive of claim 83, wherein the rounded particles comprise glass beads.

85. The abrasive of claim 83, wherein the rounded particles are hollow.

86. The abrasive of claim 83, wherein the sharp-edged particles comprise aluminum oxide particles.

87. The abrasive of claim 83, wherein the sharp-edged particles comprise sand particles.

88. The abrasive of claim 83, wherein the sharp-edged particles comprise glass particles.

89. A method of treating skin, comprising:
propelling rounded particles against the skin.

90. The method of claim 89, wherein the rounded particles comprise glass beads.

91. The method of claim 89, wherein the rounded particles comprise ceramic material.

92. The method of claim 89, wherein the rounded particles are hollow.

93. The method of claim 89, further comprising mixing the rounded particles with aluminum oxide particles prior to propelling the rounded particles against the skin.

94. The method of claim 89, further comprising mixing the rounded particles with sand particles prior to propelling the rounded particles against the skin.

95. The method of claim 89, wherein the rounded particles comprise spheroidal shaped particles.

96. The method of claim 89, wherein the rounded particles comprise substantially ellipsoidal shaped particles.

97. The method of claim 89, wherein the rounded particles comprise substantially cylindrical shaped particles.

98. The method of claim 89, wherein the rounded particles have sizes between about 25 microns and about 325 microns.

99. The method of claim 89, wherein the rounded particles have a size range distribution from about 50 microns to about 180 microns.

100. The method of claim 89, wherein the rounded particles have a size range distribution from about 90 microns to about 150 microns.

101. The method of claim 89, wherein propelling rounded particles against the skin comprises moving the rounded particles with a vacuum so that the rounded particles contact the skin.

102. The method of claim 89, wherein propelling rounded particles against the skin comprises moving the rounded particles with a compressed gas so that the rounded particles contact the skin.

103. The method of claim 102, wherein the gas is air.

104. The method of claim 102, wherein the gas is nitrogen.

105. The method of claim 89, further comprising propelling an antibacterial agent against the skin.

106. The method of claim 89, further comprising propelling a lotion against the skin.

107. The method of claim 89, wherein the rounded particles are used to treat stretch marks.

108. The method of claim 89, wherein the rounded particles are used to treat acne scarring.

109. The method of claim 89, wherein the rounded particles are used to treat wrinkles.

110. The method of claim 89, wherein the rounded particles are coated.

111. The method of claim 110, wherein the coating comprises a vitamin.

112. The method of claim 110, wherein the coating comprises a lubricity agent.

113. The method of claim 110, wherein the coating comprises a coloring agent.

114. The method of claim 110, wherein the coating comprises an antibacterial agent.
